(19) United States
(12) Patent Application Publication
(10) Pub. No.: US 2026/0325751 A1
(43) Pub. Date: Oct. 22, 2026

(54) ADVANCED INFUSION TIMERS

(71) Applicant: CareFusion 303, Inc., San Diego, CA (US)

(72) Inventors: Ehsan Ardabili MALEKI, Saint Johns, FL (US); Kathryn WILLYBIRO, Encinitas, CA (US)

(21) Appl. No.: 19/182,224

(22) Filed: Apr. 17, 2025

(51) Int. Cl. A61M 5/172; A61M 5/142; A61M 5/168

(52) U.S. Cl. CPC A61M 5/1723; A61M 5/16831; A61M 5/16877; A61M 2005/14292

(57) ABSTRACT

A medical timer device has processors and memory including instructions that, when executed by the processors, cause timer logic to set a timer for a delivery of a medication by intravenous infusion, to obtain an infusion variable, to calculate a time period for an operational parameter of the infusion based on the infusion variable and to generate an indicator about the operational parameter. An infusion pump and a method of using a medical timer device are also provided.

### CROSS-REFERENCES TO RELATED APPLICATIONS

N/A

### TECHNICAL FIELD

This application relates generally to advanced timing features for an infusion pump.

### BACKGROUND

Infusion pumps are frequently used in medical processes to deliver fluids, supplements and medications intravenously to patients. Typical infusion pumps rely on static timing or manual timing and monitoring, which can be time consuming, prone to errors, and non-responsive to clinical changes.

### SUMMARY

According to various aspects, the subject technology provides advanced timer features for infusion pumps. The advanced timer functionality may reside in and/or be provided by any of an infusion pump programmable control unit (PCU), an infusion pump module, a patient monitor, a hemodynamic monitor, a patient device in communication with an infusion pump, a sensor associated with a patient or therapy and in communication with an infusion pump, and a healthcare information system network or cloud service.

One or more embodiments of the disclosure provide for a medical timer device having one or more processors and memory including instructions that, when executed by the one or more processors, cause the medical timer device to: set, by timer logic, a timer for a delivery of a medication by intravenous (IV) infusion; obtain, by the timer logic, an infusion variable; calculate, by the timer logic, a time period for an operational parameter of the infusion based on the infusion variable; and generate, by the timer logic, an indicator about the operational parameter.

One or more embodiments of the disclosure provide for a method under control of one or more processors, the method comprising: setting, by timer logic, a timer for a delivery of a medication by intravenous (IV) infusion; obtaining, by the timer logic, an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic; calculating, by the timer logic, a time period for an operational parameter of the infusion based on the infusion variable; and generating, by the timer logic, an indicator about the operational parameter.

One or more embodiments of the disclosure provide for an infusion pump comprising: a housing; a pump motor mounted within the housing, wherein a force from the pump motor causes delivery of a fluid from a fluid source to a patient; one or more processors comprising timer logic; and memory including instructions that, when executed by the one or more processors, cause the infusion pump to: set a timer for infusion of a medication with the delivery of the fluid; obtain an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic; calculate a time period for an operational parameter of the infusion based on the infusion variable; generate an indicator about the operational parameter; and recalculate the time period for the operational parameter of the infusion based on a change in the infusion.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of an example patient care system having four fluid infusion pumps, each of which is connected to a respective fluid supply for pumping the contents of the fluid supply to a patient.

FIG. 2 depicts an example of an institutional patient care system of a healthcare organization, according to aspects of the subject technology.

FIGS. 3A and 3B depict example medical devices having timer logic, according to aspects of the subject technology.

FIG. 4 is a conceptual diagram illustrating an example electronic system for providing timer functions, according to aspects of the subject technology.

FIG. 5 is a conceptual diagram illustrating an example method of operating timer logic, according to aspects of the subject technology

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology includes a system or device that utilizes timer logic for advanced timer features of a medical device (e.g., such as an infusion pump) to optimize medication delivery and to reduce labor, time and errors by a clinician operating the infusion pump. According to some implementations, the timer logic may reside in and/or be provided by any of an infusion pump PCU, an infusion pump module, a patient monitor, a hemodynamic monitor, a patient device in communication with an infusion pump, a sensor associated with a patient or therapy and in communication with an infusion pump, and a healthcare information system network or cloud service.

The advanced timer features of the subject technology provide many benefits. For example, advanced timer features of the subject technology improve patient safety by reducing the risk of under or over infusion and delay in infusion introduced by manual timers or static timers that do not account for dynamic clinical events. Advanced timer features of the subject technology optimize infusion parameters based on drug properties, patient characteristics, and infusion setup for efficient drug delivery. Efficient drug delivery can ensure not just patient safety but also that the medical device(s) are not overused and the limited resources such as power, memory, processing bandwidth, and network bandwidth are conserved. Also, advanced timer features of the subject technology streamline the nursing workflow by automating reminders for dose adjustments, thus enhancing workflow efficiency by allowing nurses to focus more on patient care and less on manual calculations and monitoring.

Other advanced timer features of the subject technology provide for reduced medication errors because the timer functions are personalized, thus minimizing the risk of human error in adjusting infusion parameters. The advanced timer features of the subject technology provide improved compliance by prompting healthcare providers to adhere to physician orders and guidelines consistently, thus helping to maintain treatment efficacy and patient safety.

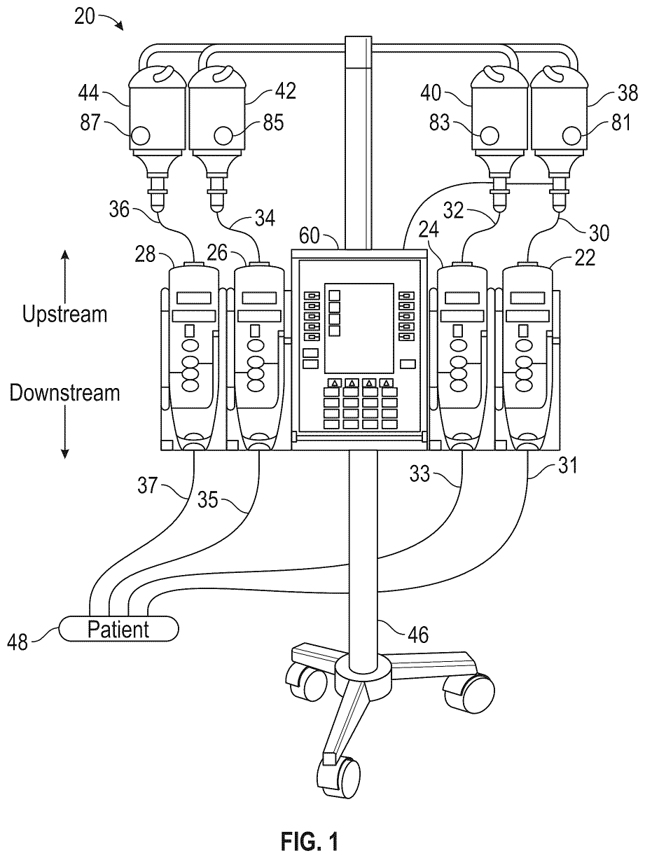

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into a patient 48. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60. In some implementations, the infusion pumps 22, 24, 26, and 28 may be configured for independent operation without the need for the pump control unit 60.

Fluid supplies 38, 40, 42, and 44 are each coupled to an electronic data tag 81, 83, 85, and 87, respectively, or to an electronic transmitter. The tag may provide information to the infusion pump or pump control unit for administering the fluid included in the respective supply. The information may include one or more of: fluid identifier, patient identifier, order identifier, or prescription identifier. A device or component associated with the infusion system may be equipped with an electronic data tag, reader, or transmitter to further provide information to the infusion pump or pump control unit to administer a therapy or fluid. In some cases, IV sets may be packaged in bags that have barcodes and/or written information that may be transmitted to the pump to provide set-related information for the timer.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

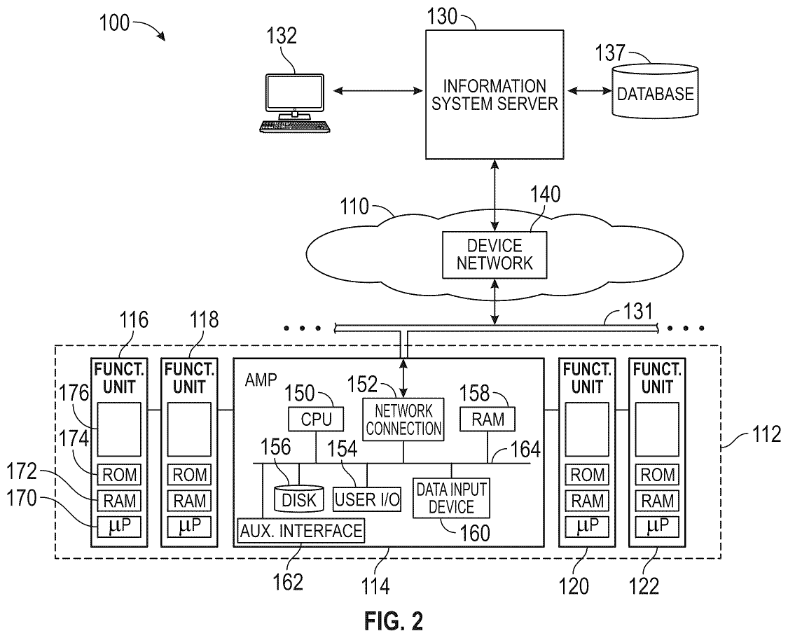

FIG. 2 depicts an example of an institutional patient care system 100 of a healthcare organization, according to aspects of the subject technology. In FIG. 2, a patient care device (or “medical device” generally) 112 is connected to a healthcare facility network 110. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each medical device 112 is connected to an internal healthcare network 110 by a transmission channel 131. Transmission channel 131 is any wired or wireless transmission channel, for example an 802.11 wireless local area network (LAN). In some implementations, network 110 also includes computer systems located in various departments throughout a healthcare facility. For example, network 110 of FIG. 2 optionally includes computer systems associated with an admissions department, a billing department, a biomedical engineering department, a clinical laboratory, a central supply department, one or more unit station computers and/or a medical decision support system. As described further below, network 110 may include discrete subnetworks. In the depicted example, network 110 includes a device network 140 by which patient care devices 112 (and other devices) communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, and mobile devices such as laptops, tablet computers, augmented reality devices, or smartphones, configured with software for communications with information system server 130 via network 110.

Patient care device 112 comprises a system for providing patient care, such as that shown in FIG. 1 or that described in U.S. Pat. No. 5,713,856 to Eggers et al., which is incorporated herein by reference for that purpose. Patient care device 112 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 112 comprises a control module or unit 114, also referred to as interface unit 114, connected to one or more functional modules 116, 118, 120, 122. The functional elements of the patient care device 112 may be included within or mounted upon a single housing or modular housing elements including connective structures to attach and detach the modules from other modules included in the patient care device 112. Interface unit 114 includes a central processing unit (CPU) 150 connected to a memory, for example, random access memory (RAM) 158, and one or more interface devices such as user interface device 154, a coded data input device 160, a network connection 152, and an auxiliary interface 162 for communicating with additional modules or devices. Interface unit 114 also, although not necessarily, includes a main non-volatile storage unit 156, such as a hard disk drive or non-volatile flash memory, for storing software and data and one or more internal buses 164 for interconnecting the aforementioned elements.

In various implementations, user interface device 154 is a touch screen for displaying information to a user and allowing a user to input information by touching defined areas of the screen. Additionally, or in the alternative, user interface device 154 could include any means for displaying and inputting information, such as a monitor, a printer, a keyboard, softkeys, a mouse, a track ball and/or a light pen. Data input device 160 may be a bar code reader capable of scanning and interpreting data printed in bar coded format. Additionally or in the alternative, data input device 160 can be any device for entering coded data into a computer, such as a device(s) for reading a magnetic strips, radio-frequency identification (RFID) devices whereby digital data encoded in RFID tags or smart labels (defined below) are captured by the reader 160 via radio waves, PCMCIA smart cards, radio frequency cards, memory sticks, CDs, DVDs, or any other analog or digital storage media. Other examples of data input device 160 include a voice activation or recognition device or a portable personal data assistant (PDA). Depending upon the types of interface devices used, user interface device 154 and data input device 160 may be the same device.

Although data input device 160 is shown in FIG. 2 to be disposed within interface unit 114, it is recognized that data input device 160 may be integral within pharmacy system 134 or located externally and communicating with pharmacy system 134 through an RS-232 serial interface or any other appropriate communication means. Auxiliary interface 162 may be an RS-232 communications interface, however any other means for communicating with a peripheral device such as a printer, patient monitor, infusion pump or other medical device may be used without departing from the subject technology. Additionally, data input device 160 may be a separate functional module, such as modules 116, 118, 120 and 122, and configured to communicate with control unit 114, or any other system on the network, using suitable programming and communication protocols.

Network connection 152 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 116, 118, 120, 122 are any devices for providing care to a patient or for monitoring patient condition. As shown in FIG. 2, at least one of functional modules 116, 118, 120, 122 may be an infusion pump module such as an intravenous infusion pump for delivering medication or other fluid to a patient. For the purposes of this discussion, functional module 116 is an infusion pump module. Each of functional modules 118, 120, 122 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 118, 120 and/or 122 may be a printer, scanner, bar code reader or any other peripheral input, output or input/output device.

Each functional module 116, 118, 120, 122 communicates directly or indirectly with interface unit 114, with interface unit 114 providing overall monitoring and control of device 112. Functional modules 116, 118, 120, 122 may be connected physically and electronically in serial fashion to one or both ends of interface unit 114 as shown in FIG. 2, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 114. As described above, additional medical devices or peripheral devices may be connected to patient care device 112 through one or more auxiliary interfaces 162.

Each functional module 116, 118, 120, 122 may include module-specific components 176, a microprocessor 170, a volatile memory 172 and a nonvolatile memory 174 for storing information. It should be noted that while four functional modules are shown in FIG. 2, any number of devices may be connected directly or indirectly to central control unit 114. The number and type of functional modules described herein are intended to be illustrative, and in no way limit the scope of the subject technology. Module-specific components 176 include any components necessary for operation of a particular module, such as a pumping mechanism for infusion pump module 116.

While each functional module may be capable of a least some level of independent operation, interface unit 114 monitors and controls overall operation of device 112. For example, as will be described in more detail below, interface unit 114 provides programming instructions to the functional modules 116, 118, 120, 122 and monitors the status of each module.

Patient care device 112 is capable of operating in several different modes, or personalities, with each personality defined by a configuration database. The configuration database may be a database on storage unit 156 internal to patient care device 112, or an external database 137. A particular configuration database is selected based, at least in part, by patient-specific information such as device or patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 112 location in the hospital or hospital computer network. Patient care information may be entered through network connection 152 or any of input/interface devices 154, 160 or 162, and may originate from anywhere in network 110, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 112 and network 110 may communicate via automated interaction, manual interaction or a combination of both automated and manual interaction. Automated interaction may be continuous or intermittent and may occur through direct network connection 154 (as shown in FIG. 2), or through RS232 links, MIB systems, RF links such as BLUETOOTH, IR links, WLANS, digital cable systems, telephone modems or other wired or wireless communication means. Manual interaction between patient care device 112 and network 110 involves physically transferring, intermittently or periodically, data between systems using, for example, user interface device 154, coded data input device 160, bar codes, computer disks, portable data assistants, memory cards, or any other media for storing data. The communication means in various aspects is bidirectional with access to data from as many points of the distributed data sources as possible. Decision-making can occur at a variety of places within network 110. For example, and not by way of limitation, decisions can be made in HIS server 130, decision support 148, remote data server 149, hospital department or unit stations 146, or within patient care device 112 itself.

All direct communications with medical devices operating on a network in accordance with the subject technology may be performed through information system server 130, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

A medical device including one or more of the features described may be implemented as an ambulatory medical device. Ambulatory medical devices generally refer to devices designed to be portable to support administration of medication during transportation of a patient or remote medication administration (e.g., outside of a health care facility such as in a user's home). U.S. Pat. No. 7,163,381 to Barak describes a pump that may be suitable for ambulatory care and modified to include the advanced timer features described to assist in providing safe administration during mobility events. The disclosure of U.S. Pat. No. 7,163,381 is incorporated by reference in its entirety.

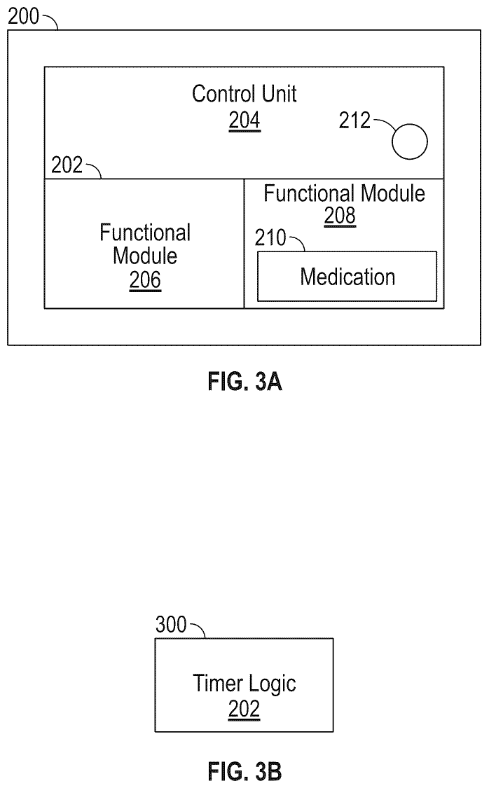

FIG. 3A depicts an example of a medical device 200 (e.g., infusion pump), in accordance with aspects of the subject technology. As shown, medical device 200 may include a control unit 204 and one or more functional modules (e.g., functional modules 116, 118, 120, 122), including a first functional module 206 having timer logic and a second functional module 208. The second functional module 208 is depicted as including a medication 210. In some implementations, medical device 200 may be a dispensing device configured to, on authorizing by a clinician, dispense the medication 210 for care of the patient. In some implementations, medical device 200 may be an infusion device configured to administer the medication 210 to the patient (e.g., intravenously by way of a connected infusion set). Control unit 204 may include one or more processors to be configured to interface with the functional units 206, 208 and to control and provide power to the functional units 206, 208 when the functional units 206, 208 are connected to the control unit 204.

According to various aspects of the subject technology, medical device 200 may include one or more sensors 212 configured to interact with the timer logic 202 in functional unit 206. Medical device 200 may be configured to measure, monitor, display and/or control, using sensor(s) 212, timer features of the medical device 200.

Often, it is desirable to know the time that the medication will actually reach the patient after an infusion is started. In one or more embodiments of the disclosure, timer logic 202 may provide a timer based on or relative to an IV set in order to inform a clinician of a time delay between a delivery start (e.g., begin infusion) and a medication reaching the patient. Here, the timer logic 202 may calculate the estimated time required for an infusate (e.g., medication 210) to travel from a source (e.g., IV bag or syringe, fluid supply 38), through the infusion pump (e.g., infusion pump 22, infusion pump module 116, functional module 208) at the designated infusion rate, through an IV tube (e.g., downstream fluid line 31) of the IV set that is coupled to the infusion pump and to the patient (e.g., patient 48).

In aspects of the disclosure, the time is updated in real time when the infusion rate is updated. In aspects of the disclosure, the dimensions (e.g., length, diameter) of the IV set or IV tube are obtained manually from direct user input and/or user input of the IV set model number. In aspects of the disclosure, the dimensions of the IV set or IV tube are obtained by the timer logic 202 recognizing the IV set by any of near field communications (NFC) technology, optical technology (e.g., sensors, readers), scanning technology (e.g., barcode, QRC) and the physical fit of the IV set into the infusion pump.

In another scenario, it is desirable to know the delivery rate of a medication to a patient. In one or more embodiments of the disclosure, timer logic 202 may provide a timer based on flow rate accuracy in order to inform a clinician of the actual delivery rate of the medication to the patient. Here, the timer logic 202 may calculate the estimated time required for the infusate to be delivered to the patient based on a programmed flow rate and any or all factors that impact flow rate accuracy, including back pressure, temperature, head height (i.e., relative distance between pump and infusate bag) and infusate viscosity. For example, an actual flow rate may equal a programmed flow rate plus a mathematical function fcn(back pressure, temperature, head height, infusate viscosity), where fcn(back pressure, temperature, head height, infusate viscosity) is a mathematical function, derived empirically and/or theoretically, that takes in a number of input parameters and outputs a variation between the actual flow rate and the programmed flow rate. This function could take many forms, including a simple proportional formula such as fcn(back pressure, temperature, head height, infusate viscosity)=A*back pressure+B*temperature+C*head height+D*infusate viscosity; or any other linear or nonlinear mathematical relationship that best describes the impact of the parameter on the infusion rate.

In aspects of the disclosure, the time is updated in real time by the timer logic 202 when the programmed infusion rate is updated. In aspects of the disclosure, the impact of each factor on the infusion rate is characterized. For example, the characterization may be a simple look-up table, a transfer function, or a more advanced mathematical model. In aspects of the disclosure, the timer logic 202 uses the values of all available factors to estimate the actual flow rate. In aspects of the disclosure, the timer logic 202 obtains the value of each factor by manual user entry and/or detection by the timer logic 202 via embedded sensors (e.g., pressure sensors, temperature sensors). In aspects of the disclosure, the time is updated in real time by the timer logic 202 when any of the factors changes.

As conditions may change during a therapy, in some implementations, the timer logic 202 may receive a stream of measurements of back pressure, temperature, or head height. As the stream of measurements is received, the timer logic 202 may calculate and cause display of an updated delivery time. In some implementations, if the updated delivery time differs from a prior delivery time by a predetermined magnitude, the timer logic 202 may transmit an alert or other message to indicate a substantial deviation from a previous expectation.

In yet another example, it is desirable to utilize infusion drug pharmacokinetics and patient characteristics for an infusion process. In one or more embodiments of the disclosure, timer logic 202 may provide a timer to optimize drug delivery based on the patient characteristics and the pharmacokinetics of the drug. Here, the timer logic 202 may use the pharmacokinetics of the drug (e.g., absorption rate, half-life, etc.) and the relevant patient characteristics (e.g., weight, age, etc.) to estimate the time it will take for the drug to affect the desired change in patient parameters and/or for the drug to reach its half-life. For example, if a drug is being titrated to reach a desired change in a patient parameter, the timer logic 202 may be used to ensure titration does not occur too fast or too slow by providing guidance to the clinician on the estimated amount of time to wait to see the impact of an ongoing infusion before electing to make a change. As another example, if a bolus infusion is given, the timer logic 202 would calculate the half-life and generate an alert when the effect of the drug is likely wearing off so the clinician can then assess the patient and determine if a second bolus is necessary.

In aspects of the disclosure, the timer logic 202 may track the change in patent parameters and/or drug half-life and alert the clinician when either scenario has been reached. In aspects of the disclosure, the time is updated in real time by the timer logic 202 when the drug type or the drug concentration is changed. In aspects of the disclosure, the drug pharmacokinetics and patient characteristics may reside within the timer logic 202 or the device that contains the timer logic 202. In aspects of the disclosure, the drug pharmacokinetics and patient characteristics may be obtained manually from user input. In aspects of the disclosure, the drug pharmacokinetics and patient characteristics may be obtained from another connected device (e.g., infusion pump, electronic medical record (EMR), etc.).

In another example, it is desirable to have timing functions based on a physician's order and/or a clinical protocol. In one or more embodiments of the disclosure, timer logic 202 may provide a timer to follow the physician's or clinical protocol's prescribed frequency for changes in dose and/or rate. Here, the timer logic 202 may provide for an input (e.g., manual, an automatic programming request (APR), a connected system) of the desired frequency (e.g., every 1 hour), based on the physician's order or clinical protocol, for dose adjustments. In aspects of the disclosure, the timer logic 202 may cause an infusion device to generate an alert when it is time to update the infusion parameters (e.g., infusion rate, drug dose, drug selection).

In another example, it is desirable to have timing functions based on one or more of arterial or venous blood pressure waveform parameters, shape parameters and waveform features. In one or more embodiments of the disclosure, timer logic 202 may inform a clinician of a time delay between a medication delivery start (e.g., infusion start) and the medication reaching the patient. Here, the timer logic 202 may use a patient's arterial and/or venous blood pressure waveform with or without relevant patient characteristics (e.g., weight, age, etc.) to inform a user (e.g., clinician) when the infused drug has reached the patient and impacted the patient's blood pressure waveform.

In aspects of the disclosure, the timer logic 202 may track and generate an alert when the infused drug has changed the patient's arterial or venous blood pressure waveform. In aspects of the disclosure, the timer logic 202 may be based on arterial or venous waveform shape parameters or a detected change in shape parameters (e.g., systolic phase parameters, diastolic phase parameters, systolic rise phase parameters, systolic decay phase parameters and overall decay phase parameters). In aspects of the disclosure, the timer logic 202 may be based on individual features of the arterial or venous blood pressure waveform or a detected change in features (e.g., signal features, features used in an industry standard algorithm, features used in a proprietary algorithm, complexity features, baroflex features, variability features, spectral features, delta change features, or power combinations of these features).

In aspects of the disclosure, the timer logic 202 may be based on standard monitored parameters or a detected change in parameters (e.g., absolute change, percent change, trend, etc.), such as systolic blood pressure, diastolic blood pressure and mean blood pressure. In aspects of the disclosure, the timer logic 202 may update the time in real time if a drug type or a drug concentration is changed.

In aspects of the disclosure, the drug pharmacokinetics and/or patient characteristics and/or blood pressure waveform may reside within the timer logic 202 and be used in combination with the patient's arterial or venous blood pressure waveform shape. In aspects of the disclosure, the drug pharmacokinetics and/or patient characteristics and/or blood pressure waveform may be obtained manually from user input. In aspects of the disclosure, the drug pharmacokinetics and/or patient characteristics and/or blood pressure waveform may be obtained from another connected device (e.g., infusion pump, EMR, etc.).

In some aspects of the disclosure, the timer logic 202 may be part of a medical device 300 as shown in FIG. 3B. For example, medical device 300 may be a patient monitor in the form of any of a finger clamp, a cuff, a skin pad, an intravenous device, one or more contact pads/leads, etc. As another example, medical device 300 may be a hemodynamic monitor to measure/monitor patient functions (e.g., ECG, heart rate, blood pressure, central venous pressure, temperature, peripheral venous oxygen saturation, blood gas analysis).

In some aspects of the disclosure, the medical device 300 may be a pump control unit (e.g., pump control unit 60) that provides for programming and controlling of one or multiple infusion pumps. In some aspects of the disclosure, the medical device 300 may be an infusion pump (e.g., infusion pump 22, infusion pump module 116). In some aspects of the disclosure, the medical device 300 may be a sensor device (e.g., pressure sensor, temperature sensor, pulse sensor). In some aspects of the disclosure, the medical device 300 may be a separate timer device.

In some aspects of the disclosure, the timer logic 202 may be part of a network/cloud service (e.g., healthcare facility network 110). Here, the timer logic 202 may be computer code, software, firmware, part of a server, etc.

In some aspects of the disclosure, the timer logic 202 may be configured to execute or provide any of the timer functions discussed above, individually or in any combination.

Computer program code for carrying out operations of the subject technology may be written in an object oriented programming language such as, for example, JAVA®, Smalltalk, or C++. However, the computer program code for carrying out operations of the subject technology may also be written in conventional procedural programming languages, such as the “C” programming language, in an interpreted scripting language, such as Perl, or in a functional (or fourth generation) programming language such as Lisp, SML, Forth, or the like. The software may also be written to be compatible with HLA-7 requirements.

According to various implementations, the medical device 200, 300 may include and/or utilize one or more sensors that are configured to measure patient parameters. The medical device 200, 300 may include circuitry and/or software that monitors the measurements and/or values received from each of these sensors. When a medical device 200, 300 (or its network circuitry) detects that one of these measurements and/or values satisfies (e.g., exceeds) a predetermined threshold, the medical device 200, 300 may be triggered to automatically adjust or change settings and/or a mode of operation or generate an alarm.

According to various implementations, adjusting or changing a mode of operation may include taking a corrective action. A corrective action may include displaying an alert on the medical device 200, 300 and/or sounding an audible alert. A corrective action may include stopping an ongoing administration of a medication or locking the device 200, 300 from administering or providing further medications until the alert has been acknowledged. In a clinical setting, acknowledgement may include identifying a clinician authorized to the medical device 200, 300 by way of the clinician scanning a badge, and the clinician manually dismissing the alert by way of a manual input at the medical device 200, 300 or by way of a computing device connected to the medical device 200, 300 (e.g., over a network). In a home setting, acknowledgement may include providing patient oriented step by step instructions to the home user (e.g., patient, home caregiver), and the home user manually dismissing the alert by way of a manual input at the medical device 200, 300 or by way of a computing device connected to the medical device 200, 300 (e.g., over a network).

Many medical devices 200, 300 may be battery operated, so that the devices 200, 300 may maintain operations in the event of a power outage or when moving between care areas. The battery unit of a medical device 200, 300 may provide power to, for example, one or more processors responsible for operations, a pumping mechanism (e.g., in the case of an infusion pump), an electronic lock (e.g., in the case of a dispensing device), or network circuitry. The battery unit may also be responsible for providing power to subordinate or secondary devices connected to the medical device 200, 300. For example, a battery unit of an infusion device may provide power to one or more connected functional modules that each include an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like.

Some environmental conditions may cause a medical device 200, 300 to lose network connectivity. For example, some clinical care areas may include shielded rooms (such as MRI rooms) that prevent radio and electromagnetic energy from passing through walls and some home setting areas may include sparsely populated areas with little or no wireless reception (e.g., vacation home in the mountains). When a medical device 200, 300 (or its network circuitry) detects it has lost network connectivity, the medical device 200, 300 may be triggered to automatically adjust or change modes. In one example, on detecting a loss of network connectivity, a medical device 200, 300 may switch to a manual mode in which it begins using an onboard drug library instead of looking up operating parameters and limits via the network connection.

In some implementations, a medical device 200, 300 may be configured to produce audible or visual alerts prior to or when changing modes. In this regard, the medical device 200, 300, on losing a network connection, may provide on its associated display device an option for mode adjustment. For example, the medical device 200, 300 may prompt a user to select whether to maintain the current mode or adjust the network setting. Options may include selecting from available networks or selecting a different type of network to use (e.g., Bluetooth or wired instead of WiFi). In some implementations, alerts may be escalated as environmental conditions worsen. For example, as more critical life-supporting systems are impacted different levels of alerts may be produced. A first alert may include a visual alert, while an audible alert may progressively come louder according to different thresholds of impact, with higher impacts leading to network messages being sent to a primary caregiver via the network.

One or more embodiments of the disclosure provide for a method under control of one or more processors, the method comprising: setting, by timer logic, a timer for a delivery of a medication by intravenous (IV) infusion; obtaining, by the timer logic, an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic; calculating, by the timer logic, a time period for an operational parameter of the infusion based on the infusion variable; and generating, by the timer logic, an indicator about the operational parameter.

In aspects of the disclosure, the infusion variable comprises one or more dimensions of an IV set used in the infusion and the operational parameter comprises travel time for a first portion of the medication from a medication source, through an infusion pump at a designated infusion rate and through the IV set. In aspects of the disclosure, the dimensions of the IV set are obtained by the timer logic based on one of direct manual input of the dimensions, input of an identifier of the IV set and recognition of the IV set by one of a near field communication input, an optical input, a barcode input, a two-dimensional scan code (e.g., QRC) input and a physical fit of the IV set into the infusion pump.

In aspects of the disclosure, the infusion variable comprises a flow rate accuracy of the infusion and the operational parameter comprises a programmed flow rate of an infusion pump and one or more factors that impact flow rate accuracy comprising back pressure, temperature, head height and infusate viscosity. In aspects of the disclosure, an impact of each of the one or more factors is characterized based on one of a look up table, a transfer function and a mathematical model. In aspects of the disclosure, the timer logic uses values of all available factors to calculate an actual flow rate. In aspects of the disclosure, a value of each factor is obtained by the timer logic based on one of direct manual input and receipt of a measurement from a sensor.

In aspects of the disclosure, the infusion variable comprises one or more pharmacokinetics of the medication and one or more patient characteristics and the operational parameter comprises at least one of the medication affecting an intended change in patient parameters and a half-life of the medication. In aspects of the disclosure, the timer logic generates an alert when either the medication affects the intended change in patient parameters and the half-life of the medication is reached. In aspects of the disclosure, the one or more pharmacokinetics of the medication and one or more patient characteristics are one of stored within the medical timer device, obtained by direct manual input and received from a connected device.

In aspects of the disclosure, the infusion variable comprises a prescribed frequency for changes in one of a medication type, a medication dosage and an infusion rate and the operational parameter comprises an alert generation. In aspects of the disclosure, the timer logic generates an alert when a time to make a prescribed change is reached.

In aspects of the disclosure, the infusion variable comprises one of a blood pressure parameter of a patient and patient characteristics and the operational parameter comprises the medication reaching the patient and a resulting change in the blood pressure parameter of the patient. In aspects of the disclosure, the timer logic generates an alert when the change in the blood pressure parameter of the patient occurs based on the medication. In aspects of the disclosure, the blood pressure parameter comprises one of an arterial waveform shape parameter, a venous waveform shape parameter, a detected change in a waveform shape parameter, a systolic phase parameter, a diastolic phase parameter, a systolic rise phase parameter and a systolic decay phase parameter. In aspects of the disclosure, the blood pressure parameter comprises one of a feature of a blood pressure waveform and a detected change in the feature of the blood pressure waveform, wherein the feature comprises one of a signal feature, a feature used in a proprietary algorithm, a complexity feature, a baroflex feature, a variability feature and a spectral feature. In aspects of the disclosure, one or more of a pharmacokinetic of the medication, a patient characteristic and a blood pressure waveform are one of stored within the medical timer device, obtained by direct manual input and are obtained by a connected device.

In aspects of the disclosure, the time period is recalculated by the timer logic in real time based on one of a change to an infusion rate from a designated infusion rate, a change to the operational parameter, a change to a medication type for the infusion and a change to a medication concentration for the infusion.

One or more embodiments of the disclosure provide for a method under control of one or more processors, the method comprising: setting, by timer logic, a timer for a delivery of a medication by intravenous (IV) infusion; obtaining, by the timer logic, an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic; calculating, by the timer logic, a time period for an operational parameter of the infusion based on the infusion variable; and generating, by the timer logic, an indicator about the operational parameter.

One or more embodiments of the disclosure provide for an infusion pump comprising: a housing; a pump motor mounted within the housing, wherein a force from the pump motor causes delivery of a fluid from a fluid source to a patient; one or more processors comprising timer logic; and memory including instructions that, when executed by the one or more processors, cause the infusion pump to: set a timer for infusion of a medication with the delivery of the fluid; obtain an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic; calculate a time period for an operational parameter of the infusion based on the infusion variable; generate an indicator about the operational parameter; and recalculate the time period for the operational parameter of the infusion based on a change in the infusion.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

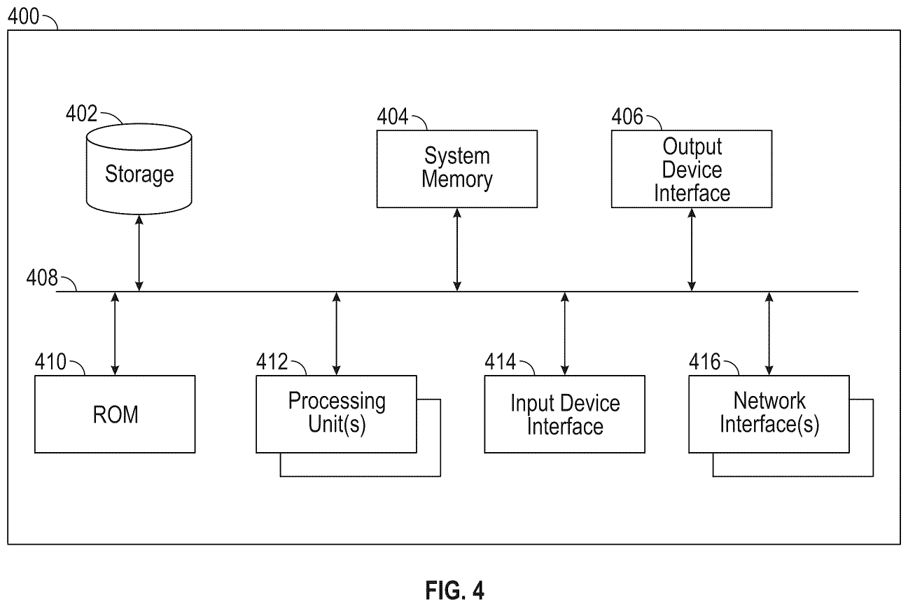

FIG. 4 is a conceptual diagram illustrating an example electronic system 400 for automatically adapting control of a medical device responsive to detecting a location setting change, according to aspects of the subject technology. Electronic system 400 may be a computing device for execution of software associated with one or more components and processes provided by FIGS. 1-3, including but not limited to information system server 130, device terminal 132, computing hardware within patient care device 112, or external database 137. Electronic system 400 may be representative, in combination with the disclosure regarding FIGS. 1-3. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 614, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

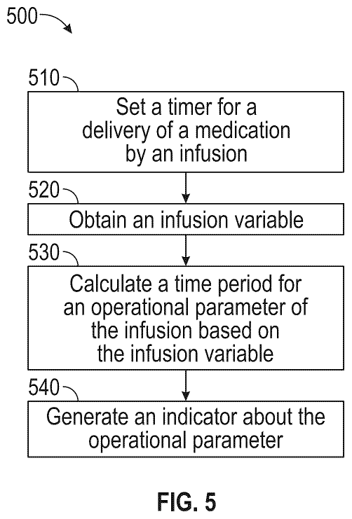

As shown in FIG. 5, a method 500 is provided for operating timer logic (e.g., timer logic 202) under control of one or more processors (e.g., processing unit(s) 412) during an infusion. Step 510 is setting, by the timer logic, a timer for a delivery of a medication by IV infusion. Step 520 is obtaining, by the timer logic, an infusion variable. Here, the infusion variable may be any of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic. Step 530 is calculating, by the timer logic, a time period for an operational parameter of the infusion based on the infusion variable. Step 540 is generating, by the timer logic, an indicator about the operational parameter.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

The functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

Various devices described may “provide” messages, alerts, alarms, power, interfaces or other operational outputs. The use of these terms (e.g., “provide” or “providing”) in this manner encompasses a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A medical timer device comprising:
one or more processors; and
memory including instructions that, when executed by the one or more processors, cause the medical timer device to:
set, by timer logic, a timer for a delivery of a medication by intravenous (IV) infusion;
obtain, by the timer logic, an infusion variable;
calculate, by the timer logic, a time period for an operational parameter of the infusion based on the infusion variable; and
generate, by the timer logic, an indicator about the operational parameter.

2. The medical timer device of claim 1, wherein the infusion variable comprises one or more dimensions of an IV set used in the infusion and the operational parameter comprises travel time for a first portion of the medication from a medication source, through an infusion pump at a designated infusion rate and through the IV set.

3. The medical timer device of claim 2, wherein the dimensions of the IV set are obtained by the timer logic based on one of:
direct manual input of the dimensions;
input of an identifier of the IV set; and
recognition of the IV set by one of a near field communication input, an optical input, a barcode input, a two-dimensional scan code input and a physical fit of the IV set into the infusion pump.

4. The medical timer device of claim 1, wherein the infusion variable comprises a flow rate accuracy of the infusion and the operational parameter comprises a programmed flow rate of an infusion pump and one or more factors that impact flow rate accuracy comprising back pressure, temperature, head height and infusate viscosity.

5. The medical timer device of claim 4, wherein an impact of each of the one or more factors is characterized based on one of a look up table, a transfer function and a mathematical model.

6. The medical timer device of claim 4, wherein the timer logic uses values of all available factors to calculate an actual flow rate.

7. The medical timer device of claim 4, wherein a value of each factor is obtained by the timer logic based on one of:
direct manual input; and
receipt of a measurement from a sensor.

8. The medical timer device of claim 1, wherein the infusion variable comprises one or more pharmacokinetics of the medication and one or more patient characteristics and the operational parameter comprises at least one of the medication affecting an intended change in patient parameters and a half-life of the medication.

9. The medical timer device of claim 8, wherein the timer logic generates an alert when either the medication affects the intended change in patient parameters and the half-life of the medication is reached.

10. The medical timer device of claim 8, wherein the one or more pharmacokinetics of the medication and one or more patient characteristics are one of:
stored within the medical timer device;
obtained by direct manual input; and
received from a connected device.

11. The medical timer device of claim 1, wherein the infusion variable comprises a prescribed frequency for changes in one of a medication type, a medication dosage and an infusion rate and the operational parameter comprises an alert generation.

12. The medical timer device of claim 11, wherein the timer logic generates an alert when a time to make a prescribed change is reached.

13. The medical timer device of claim 1, wherein the infusion variable comprises one of a blood pressure parameter of a patient and patient characteristics and the operational parameter comprises the medication reaching the patient and a resulting change in the blood pressure parameter of the patient.

14. The medical timer device of claim 13, wherein the timer logic generates an alert when the change in the blood pressure parameter of the patient occurs based on the medication.

15. The medical timer device of claim 13, wherein the blood pressure parameter comprises one of:
an arterial waveform shape parameter;
a venous waveform shape parameter;
a detected change in a waveform shape parameter;
a systolic phase parameter;
a diastolic phase parameter;
a systolic rise phase parameter; and
a systolic decay phase parameter.

16. The medical timer device of claim 13, wherein the blood pressure parameter comprises one of a feature of a blood pressure waveform and a detected change in the feature of the blood pressure waveform, wherein the feature comprises one of:
a signal feature;
a feature used in a proprietary algorithm;
a complexity feature;
a baroflex feature;
a variability feature; and
a spectral feature.

17. The medical timer device of claim 13, wherein one or more of a pharmacokinetic of the medication, a patient characteristic and a blood pressure waveform are one of:
stored within the medical timer device;
obtained by direct manual input; and
are obtained by a connected device.

18. The medical timer device of claim 1, wherein the time period is recalculated by the timer logic in real time based on one of:
a change to an infusion rate from a designated infusion rate;
a change to the operational parameter;
a change to a medication type for the infusion; and
a change to a medication concentration for the infusion.

19. A method comprising:
under control of one or more processors,
setting, by timer logic, a timer for a delivery of a medication by intravenous (IV) infusion;
obtaining, by the timer logic, an infusion variable comprising one of:
a dimension of an IV set used in the infusion;
a flow rate accuracy of the infusion;
a pharmacokinetic of a medication for the infusion and a patient characteristic;
a prescribed frequency for a change in one of a medication type, a medication dosage and an infusion rate; and
a blood pressure parameter of a patient and a patient characteristic;
calculating, by the timer logic, a time period for an operational parameter of the infusion based on the infusion variable; and
generating, by the timer logic, an indicator about the operational parameter.

20. An infusion pump, comprising:
a housing;
a pump motor mounted within the housing, wherein a force from the pump motor causes delivery of a fluid from a fluid source to a patient;
one or more processors comprising timer logic; and
memory including instructions that, when executed by the one or more processors, cause the infusion pump to:
set a timer for infusion of a medication with the delivery of the fluid;
obtain an infusion variable comprising one of a dimension of an IV set used in the infusion, a flow rate accuracy of the infusion, a pharmacokinetic of a medication for the infusion and a patient characteristic, a prescribed frequency for a change in one of a medication type, a medication dosage and an infusion rate, and a blood pressure parameter of a patient and a patient characteristic;
calculate a time period for an operational parameter of the infusion based on the infusion variable;
generate an indicator about the operational parameter; and
recalculate the time period for the operational parameter of the infusion based on a change in the infusion.
